SEQUENCE LISTING (14 sequences)

SEQ ID NO: 1 - protein, 482 aa, Artificial Sequence
  Description of Artificial Sequence Synthetic polypeptide
GVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 2 - protein, 17 aa, Artificial Sequence
  Description of Artificial Sequence Synthetic peptide
NHLGDMTSEEVMSLTSS

SEQ ID NO: 3 - protein, 453 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYE

SEQ ID NO: 4 - protein, 1922 aa, Artificial Sequence
  Description of Artificial Sequence Synthetic polypeptide
GVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 5 - protein, 1938 aa, Artificial Sequence
  Description of Artificial Sequence Synthetic polypeptide
FEGFSFLAFEDFVSSIGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 6 - protein, 498 aa, Artificial Sequence
  Description of Artificial Sequence Synthetic polypeptide
FEGFSFLAFEDFVSSIGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 7 - protein, 1936 aa, Artificial Sequence
  Description of Artificial Sequence Synthetic polypeptide
EWCEYLGGYLRCYAGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 8 - protein, 496 aa, Artificial Sequence
  Description of Artificial Sequence Synthetic polypeptide
EWCEYLGGYLRCYAGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 9 - protein, 5 aa, Artificial Sequence
  Description of Artificial Sequence Synthetic peptide
VPGXG

SEQ ID NO: 10 - protein, 25 aa, Artificial Sequence
  Description of Artificial Sequence Synthetic peptide
GGGGSGGGGSGGGGSGGGGSGGGGS

SEQ ID NO: 11 - protein, 25 aa, Artificial Sequence
  Description of Artificial Sequence Synthetic peptide
EAAAKEAAAKEAAAKEAAAKEAAAK

SEQ ID NO: 12 - protein, 2000 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 13 - protein, 16 aa, Artificial Sequence
  Description of Artificial Sequence Synthetic peptide
FEGFSFLAFEDFVSSI

SEQ ID NO: 14 - protein, 14 aa, Artificial Sequence
  Description of Artificial Sequence Synthetic peptide
EWCEYLGGYLRCYA

(19) United States
(12) Patent Application Publication
(10) Pub. No.: US 2015/0238431 A1
(43) Pub. Date: Aug. 27, 2015

(54) ICAM-1 TARGETING ELPS

(71) Applicants: Sarah F. Hamm-Alvarez, Pasadena, CA (US); John Andrew MacKay, Pasadena, CA (US); Pang-Yu Hsueh, San Gabriel, CA (US)

(72) Inventors: Sarah F. Hamm-Alvarez, Pasadena, CA (US); John Andrew MacKay, Pasadena, CA (US); Pang-Yu Hsueh, San Gabriel, CA (US)

(73) Assignee: UNIVERSITY OF SOUTHERN CALIFORNIA, Los Angeles, CA (US)

(21) Appl. No.: 14/684,162

(22) Filed: Apr. 10, 2015

Related U.S. Application Data
(63) Continuation of application No. PCT/US2013/064718, filed on Oct. 11, 2013.
(60) Provisional application No. 61/713,426, filed on Oct. 12, 2012.

(51) Int. Cl. A61K 9/50; A61K 38/10; C07K 7/08; C07K 14/78

(52) U.S. Cl. CPC A61K 9/5052; C07K 14/78; A61K 38/10; C07K 7/08; A61K 48/00

(57) ABSTRACT

Provided herein are therapeutic agents comprising an elastin-like peptide (ELP) component and a ligand that specifically targets and binds an ICAM-1 receptor. In one aspect, the ELP comprises, or alternatively consists essentially of, or yet further consists of one or more sequence(s) designated S48I48 and/or mICMA-1 SI and/or hICAM-1 S1 or biological equivalents thereof. In a further aspect, the ELP/ligand composition further comprises a therapeutic agent. In one aspect, the therapeutic agent is specific to the treatment or amelioration of symptoms associated with autoimmune disorders such as SjS.

### CROSS-REFERENCE TO RELATED APPLICATIONS

This application is a continuation of International Application No. PCT/US2013/064718, filed Oct. 11, 2013, which in turn claims priority under 35 U.S.C.§119(e) to U.S. Provisional Application No. 61/713,426, filed Oct. 12, 2012, the contents of which are hereby incorporated by reference into the present disclosure.

### STATEMENT OF GOVERNMENT SUPPORT

This invention was made with government support under Grant Nos. EY017293, EY017293-04s1 and EY011386, awarded by the National Institutes of Health. Accordingly, the government has certain rights in the invention.

### BACKGROUND

Autoimmune diseases arise from an overactive immune response of the body against substances and tissues normally present in the body in which the body actually attacks its own cells. The immune system mistakes some part of the body as a pathogen and attacks it. This may be restricted to certain organs (e.g., in thyroiditis) or involve a particular tissue in different places (e.g., Goodpasture's disease which may affect the basement membrane in both the lung and the kidney). The treatment of autoimmune diseases is typically with immunosuppression such as with medications that decrease the immune response. The mechanisms of autoimmune diseases are not well understood and the treatment options are limited. For example, autoimmune diseases include Coeliac disease, diabetes mellitus type 1 (IDDM), systemic lupus erythematosus (SLE), Sjögren's syndrome, Churg-Strauss Syndrome, Hashimoto's thyroiditis, Graves' disease, idiopathic thrombocytopenic purpura, and rheumatoid arthritis (RA).

Sjögren's syndrome (SjS) is a chronic autoimmune inflammatory disease characterized by lymphocytic infiltration and destruction of lacrimal glands (LG) and salivary gland function (SG). SjS can occur independently (primary SjS) or in conjunction with another autoimmune disease (secondary SjS); both forms may progress to systemic disease of other organs. In both primary and secondary SjS, the presenting symptoms of ocular surface dryness, corneal irritation and increased susceptibility to infection overlap with symptoms of simple keratoconjunctivitis sicca (KCS). Despite the potentially unique disease profile that is likely to be manifested in the tears of primary and secondary SjS patients, no tear biomarkers have been established as diagnostic for either form.

Since the etiology of SjS is still elusive, the treatment is largely based on management of disease symptoms. The conventional treatment is to reduce the harmful effects of dryness, prevent from complications such as infection, and decrease discomfort. Tear or saliva substitutes are often used as lubricants to moisture the eye and mouth. Local and systemic stimulators are used to increase the secretion of lacrimal glands and saliva glands. In some cases, cholinergic agents, including pilocarpin and cevimeline, are promising for patients with residual salivary function. Ophthalmic cyclosporine is used to reduce T-cell activation and decrease lacrimal gland inflammation. Systemic Immunosuppressives are reserved for treating systemic manifestations of SjS. Nonsteroidal anti-inflammatory drugs (NSAIDs) and androgen analogues are used in combination with regular treatment.

In the past few decades, research has focused on drug loaded nanoparticles, such as liposomes, micelles, dendrimers and polymersomes. Relatively few drug carriers have been approved for use in humans, which suggests that better strategies and materials may be required to generate successful nanomedicines. Traditional drug delivery systems have a number of deficiencies including a lack of targeted delivery, high toxicity, low cellular uptake, and poor biocompatibility. Therefore, there is a need in the art for improved drug delivery systems targeted to parts of the eye.

### SUMMARY

To develop new treatments for diseases of the lacrimal gland, new drug vehicles are required that are biocompatible, biodegradable and easily modified with bioactive peptides. An emerging approach to this challenge employs genetically engineered polypeptides to drive the assembly of nanostructures. Elastin-like-polypeptides (ELPs) possess unique phase transition behavior, that mediates self-assembly of nanoparticles.

ELPs are the repeats of fragment derived from human elastin and thus are biocompatible, biodegradable, and less immunogenic. Self-assembling ELP fusions are synthesized efficiently using recombinant DNA technology and recursive directional ligation (RDL). The size, shape, and transition temperature of ELP can be easily modified by change the number of pentameric repeats, composition of the repeat, and guest amino acids.

To target the ligand to the appropriate tissue, the ICAM-1 receptor was chosen. The ICAM-1 receptor has an inflammation-induced expression pattern, which allows selectively targeting of diseased tissue. ICAM-1 receptors are internalized by endocytosis, which allows highly efficient internalization of anti-ICAM ELP protein polymer nanoparticles to the interior of diseased cells.

To that end, disclosed herein are novel methods and compositions for targeting therapeutics to specific cells or tissue. One aspect relates to a therapeutic agent comprising, or alternatively consisting essentially of, or yet further consisting of, an elastin-like peptide (ELP) component and a ligand that specifically targets and binds an ICAM-1 receptor. In one aspect, the ELP comprises, or alternatively consists essentially of, or yet further consists of one or more sequence(s) designated S48I48 (G(VPGSG)n(VPGIG)nY (SEQ ID NO: 4) (wherein n is an integer that denotes the number of repeats, and can be from about 6 to about 192, or alternatively from about 15 to 75, or alternatively from about 40 to 60, or alternatively from about 45 to 55, or alternatively about 48, e.g., S48I48 (G(VPGSG)₄₈(VPGIG)₄₈Y (SEQ ID NO: 1), wherein the integer “48” intends the number of repeats) and/or mICMA-1 SI and/or hICAM-1 S1 or biological equivalents thereof. In a further aspect, the ELP/ligand composition further comprises, or alternatively consists essentially of, or yet further consist of, a therapeutic agent. In one aspect, the therapeutic agent is specific to the treatment or amelioration of symptoms associated with autoimmune disorders such as SjS. In one embodiment, the therapeutic agent is trapped within a stable nanoparticle formed by the ELP when the environmental temperature is above the transition temperature of the ELP.

In certain embodiments, the ligand will target any cell or tissue that expresses an ICAM-1 receptor. Non-limiting examples include liver, heart, lacrimal gland, salivary gland, lung, brain, pancreatic acinar tissue, prostate or mucosal cells. In a related embodiment, the cell is a lacrimal acinar cell of the lacrimal gland.

A further aspects relates to a method for delivering a therapeutic agent comprising an elastin-like peptide (ELP) to a cell or tissue, said method comprising, or alternatively consisting essentially of, or yet further consisting of administering an elastin-like peptide (ELP) component and a ligand that specifically targets and binds an ICAM receptor wherein the ELP/ligand further comprises, or alternatively consists essentially of, or yet further consists of a therapeutic agent. In one aspect, the ELP comprises, or consists essentially of, or yet further consists of one or more sequence(s) or the group designated S48I48 (G(VPGSG)n(VPGIG)nY (SEQ ID NO: 4) (wherein n is an integer that denotes the number of repeats, and can be from about 6 to about 192, or alternatively from about 15 to 75, or alternatively from about 40 to 60, or alternatively from about 45 to 55, or alternatively about 48, e.g., S48I48 (G(VPGSG)₄₈(VPGIG)₄₈Y (SEQ ID NO: 1S1), wherein the integer “48” intends the number of repeats) and/or mICMA-1 SI and/or hICAM-1 S1 or biological equivalents of each thereof. In one aspect, the therapeutic agent is specific to the treatment or amelioration of symptoms associated with autoimmune disorders such as Sj S. A non-limiting example of a therapeutic agent peptide is a cathepsin S inhibitory peptides (CATSIP) that comprise, or alternatively consist essentially of, or yet further consist of the sequence NHLGDMTSEEVMSLTSS (SEQ ID NO: 2) or a biological equivalent thereof. The polynucleotide encoding the therapeutic agent (and therefore the polypeptide upon expression of the polynucleotide) can be fused to the N-terminal or C-terminal end of the ELP-ligand combination. In one aspect, it is fused to the N-terminal end. The administration can be in vitro or in vivo. In one embodiment, the therapeutic agent is trapped within a stable nanoparticle formed by the ELP when the environmental temperature is above the transition temperature of the ELP.

In certain embodiments, the ligand will target any cell or tissue that expresses an ICAM-1 receptor. Non-limiting examples include liver, heart, lacrimal gland, salivary gland, lung, brain, pancreatic acinar tissue, prostate or mucosal cells. In a related embodiment, the cell is a lacrimal acinar cell of the lacrimal gland.

In yet another aspect, provided is a method for treating an autoimmune disease such as SJS or a related disorder comprising, or alternatively consisting essentially of, or yet further consisting of, administering to a patient in need of such treatment an elastin-like peptide (ELP) component and a ligand that specifically targets and binds an ICAM receptor wherein the ELP/ligand further comprises, or alternatively consists essentially of, or yet further consists of a therapeutic agent. In one aspect, the ELP comprises, or consists essentially of, or yet further consists of one or more sequence(s) or the group designated S48I48 (G(VPGSG)n(VPGIG)nY (SEQ ID NO: 4) (wherein n is an integer that denotes the number of repeats, and can be from about 6 to about 192, or alternatively from about 15 to 75, or alternatively from about 40 to 60, or alternatively from about 45 to 55, or alternatively about 48, e.g., S48I48 (G(VPGSG)₄₈(VPGIG)₄₈Y (SEQ ID NO: 1), wherein the integer “48” intends the number of repeats) and/or mICMA-1 SI and/or hICAM-1 S1 or biological equivalents thereof. In one embodiment, the therapeutic agent is trapped within a stable nanoparticle formed by the ELP when the environmental temperature is above the transition temperature of the ELP. In one aspect, the therapeutic agent is specific to the treatment or amelioration of symptoms associated with an autoimmune disease, such as Sjögren's syndrome, autoimmune exocrinopathy, diabetic retinopathy, graft versus host disease, exocrinopathy, retinal venous occlusions, retinal arterial occlusion, macular edema, postoperative inflammation, uveitis retinitis, proliferative vitreoretinopathy, glaucoma, keratoconjunctivitis sicca (dry eye), scleritis or glaucoma. A non-limiting example of a therapeutic agent peptide that treats or ameliorates the symptoms of SjS is a cathepsin S inhibitory peptide (CATSIP), which comprises, or alternatively consists essentially of, or yet further consists of the sequence NHLGDMTSEEVMSLTSS (SEQ ID NO: 2) or a biological equivalent thereof.

In certain embodiments, the ligand will target any cell or tissue that expresses an ICAM-1 receptor. Non-limiting examples include liver, heart, lacrimal gland, salivary gland, lung, brain, pancreatic acinar tissue, prostate or mucosal cells. In a related embodiment, the cell is a lacrimal acinar cell of the lacrimal gland.

Kits for performing these methods are also provided. Screens are also provided

Further provided is an isolated polynucleotide encoding an agent as described herein and optionally being operatively linked to a regulatory or expression sequence. Also disclosed is an isolated vector and/or host cell comprising the isolated polynucleotide.

Yet further provided are compositions comprising a carrier, such as a pharmaceutically acceptable carrier, and one or more of an agent, a polynucleotide, a vector or a host cell.

A method for recombinantly producing a therapeutic agent is further disclosed comprising growing the host cell as described herein or expressing the polynucleotide as described herein under conditions that favor expression of the polynucleotide. In one aspect, the agent is further isolated.

### BRIEF DESCRIPTION OF THE FIGURES

FIG. 1 shows SDS-PAGE Copper stained gel of ELPs with or without ICAM-1 targeting tags. Both plain and ICAM-1-tagged ELPs were purified by inverse transition cycling (ITC), denatured using Dithiothreitol (DTT), resolved by SDS-PAGE with 50 μ g of proteins per lane, and stained with copper chloride. The purity of each ELP was analyzed by Image J, indicative of 100%, 92.9%, and 97.2%, in order, for Lane 1, 2, and 3. Lane 1, SI; Lane 2, mICAM-1 SI; Lane 3, hICAM-1 SI.

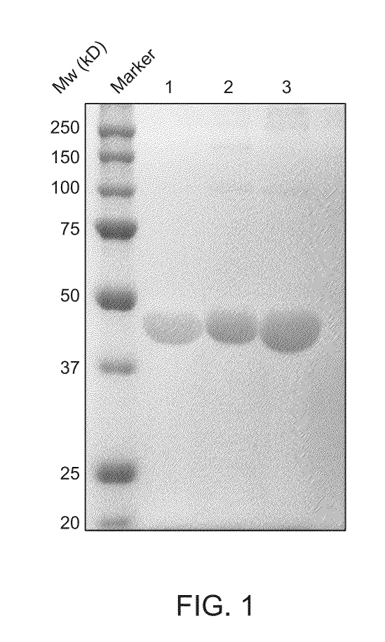

FIGS. 2A-2D show ICAM-1 targeting peptides have minimal effects on the critical micelle temperature of the ELP S48I48. FIGS. 2A and 2B show mICAM-1 SI versus SI. FIGS. 2C and 2D show hICAM-1 SI versus SI. Each sample was diluted to 25 μ M at 4° C. in PBS and passed through a 20 nm micro filter. Optical density measurements at the 350nm wavelength were used to characterize the temperature-concentration phase transitions of S48I48, mouse ICAM-1 targeting S48I48, and human ICAM-1 targeting S48I48. Human ICAM-1 targeting S48I48 tends to form big aggregates at 37°C. in PBS which suggests its unsuitability as a nano-scale drug delivery vehicle.

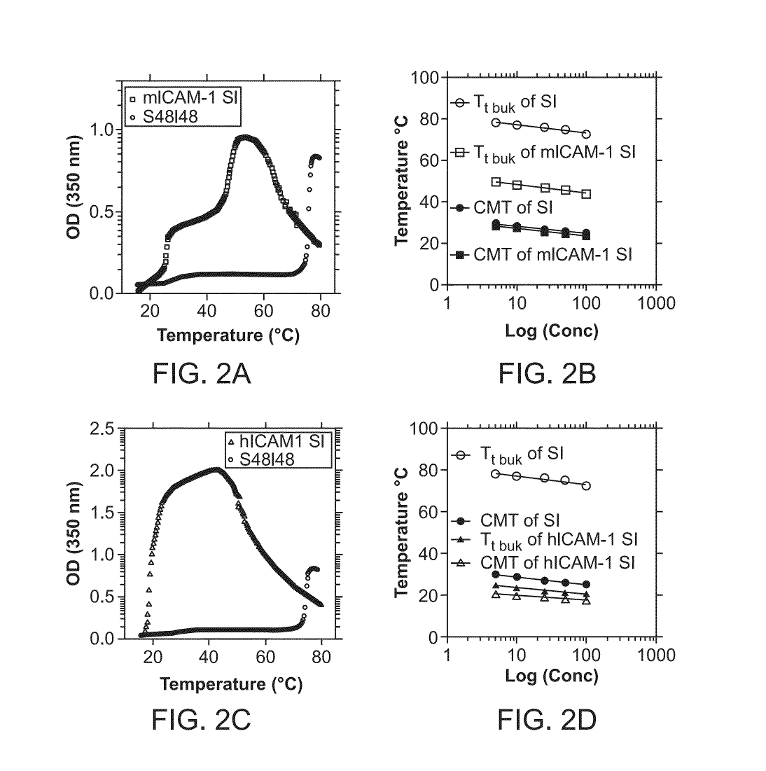

FIG. 3 shows the self-assembly and disassembly of mICAM-1 SI by changing temperature. Dynamic light scattering was used to characterize the self-assembly and disassembly of SI and mICAM-1 SI which indicate that the hydrodynamic radius (Rh) is 23.7 nm and 23.2 nm (mean+−SD), respectively, at 37° C.

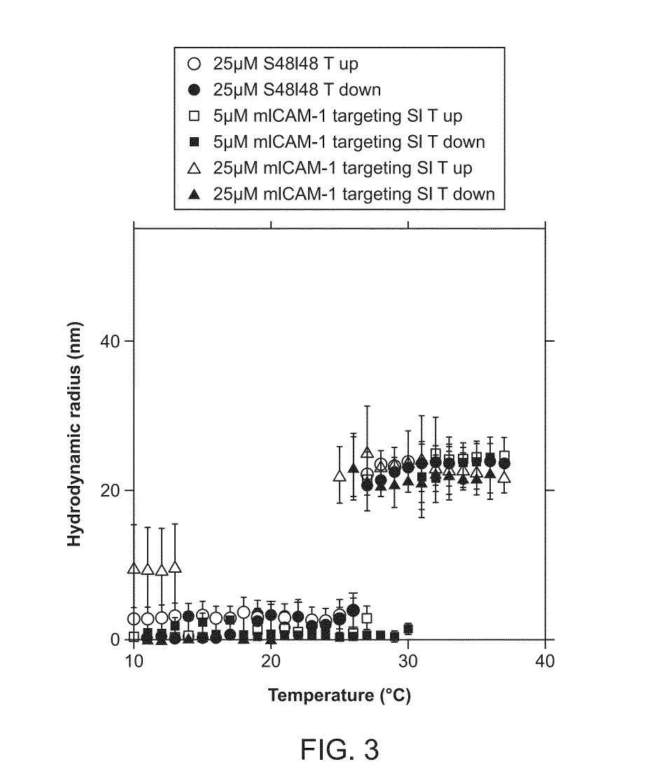

FIGS. 4A and 4B show when ICAM-1 receptor is expressed in inflamed lacrimal glands of murine model. 12-week-old male NOD mice were used as a model for inflammation in comparison with sex- and age-matched wild type (WT), BALB/C strain. FIG. 4A shows when ICAM-1 gene expression of LGs was examined by real-time PCR and the data are expressed as fold change of the expression levels against housekeeping genes, SDHA and GAPDH, respectively. FIG. 4B shows Western blot analysis shows the enhanced expression of mouse ICAM-1 in LG lysates from disease mice compared to WT controls. 20 μg of total proteins per sample were loaded into 10% SDS-PAGE. The mouse ICAM-1 receptor was present at approximately 100 kD.

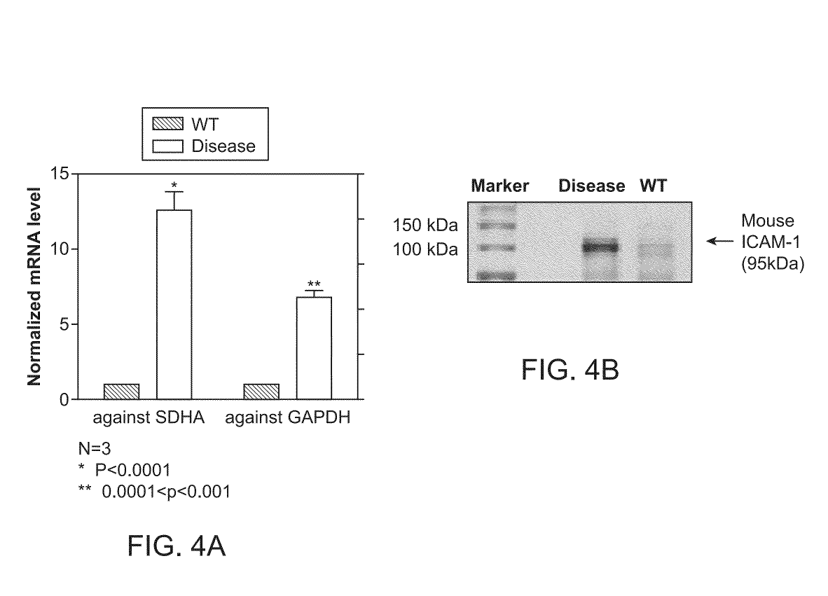

FIG. 5 shows upregulated ICAM-1 receptor distributed to the basal membrane of LGACs in NOD mice. Cryosections of LGs, derived from 12-week-old male BALB/c and NOD mice, were fixed and stained with goat anti-mouse ICAM-1 antibody followed by AF 488-conjugated anti-goat secondary antibodies (green) or treated with anti-goat secondary antibodies alone as control groups. F-actin was labeled with rhodamine phalloidin (red). Nuclei were counterstained with DAPI (blue). Overexpressed ICAM-1 receptors were localized in the basal membrane of LGACs which was shown in yellow. White arrows and arrowheads point out the basal and apical membrane of LGACs, respectively. Red arrowheads highlight infiltrating lymphocytes. Scale bar represents 10 μm.

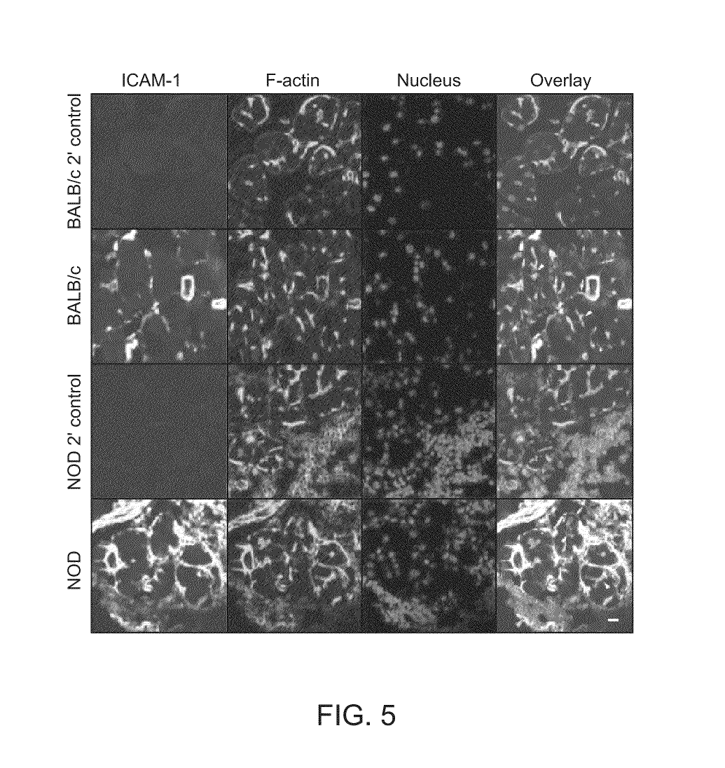

FIG. 6 shows internalized mICAM-1 SI displayed a reticular-like pattern in mICAM-1-expressing HeLa cells. HeLa cells were transfected with expression plasmids encoding full-length mouse ICAM-1-tGFP using Lipofectamine 2000. After 48-hour post transfection, HeLa cells were incubated with either 30 μM Rhodamine(Rh)-labeled SI or 30 μM Rh-mICAM-1 SI at 37° C. for 30 min and 120 min before imagined using confocal fluorescence microscopy. Unlike Rh-SI, shown weak uptake in transfected HeLa cells, Rh-mICAM-1 SI exhibited obviously more internalization via tubular or ring-like structures. Scale bar represents 10 μm.

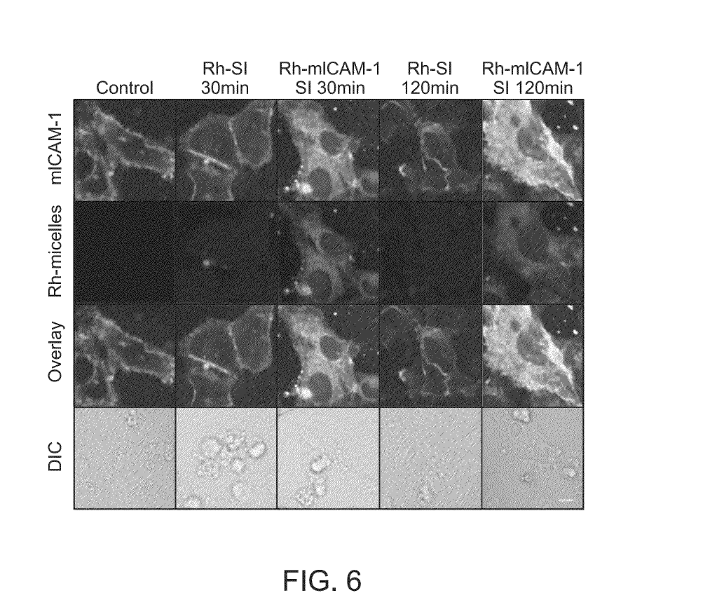

FIG. 7 shows mICAM-1 SI was internalized by human ICAM-1 receptors in HeLa cells. HeLa cells were preincubated with 20 ug/ml anti-human ICAM-1 antibody at 37° C. for 30 mins to saturate endogenous human ICAM-1 receptors. After the 30-minute pre-incubation, HeLa cells were incubated with 30 μM Rhodamineconjugated mICAM-1 SI at 37° C. for 60 min, resined with warm PBS, refreshed with medium containing 70 nM Lysotracker Green, and then imagined by confocal microscope. Scale bar represents 10μm.

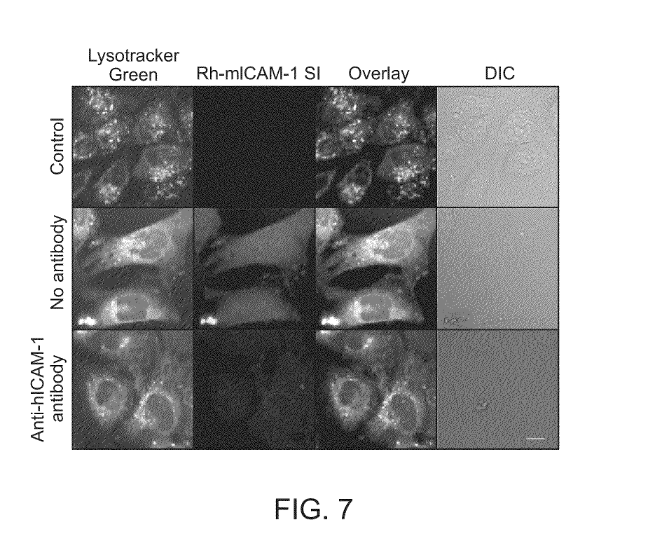

FIG. 8 shows MALDI-TOF mass spectrum of the purified S48I48, mICAM-1 SI, and hICAM-1 SI. The matrix consisted of 3,5-dimethoxy-4—hydroxycinnaminic acid (Sinapinic Acid, 50 mg/ml) dissolved in acetonitrile—0.3% acqueous trifluoroacetic acid (50:50, v/v) and the aldolase (39.21 kD) was used as an internal control.

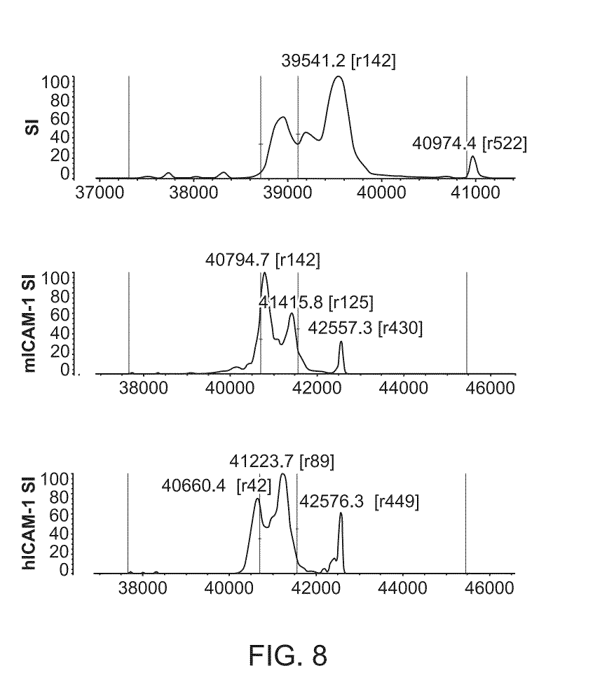

FIG. 9 shows live cell imaging for uptake of SI and mICAM-1 SI in HeLa cells.

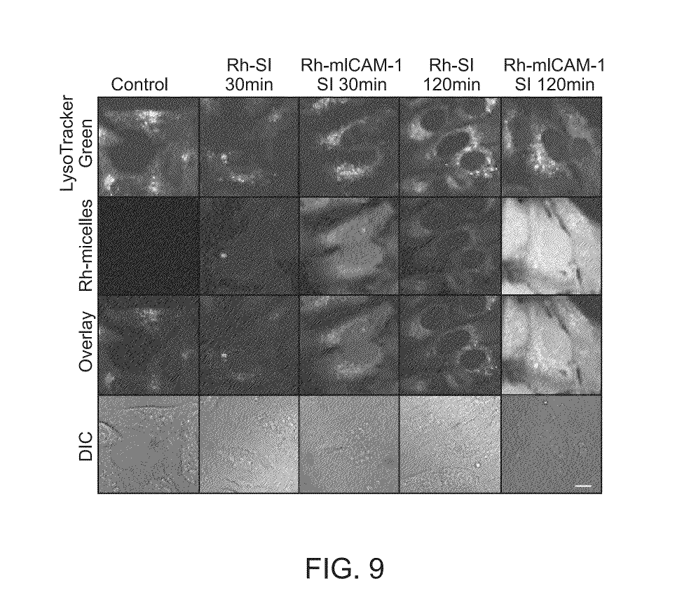

FIG. 10 represents immunostaining of HeLa cells with goat anti-human ICAM-1 antibody. (Non-transfected) HeLa cells were grown on sterile glass coverslips at 37° C. overnight. On the following day, HeLa cells were fixed with 4% paraformaldehyde for 15 min, quenched with 50 mM NH4Cl for 5 min, and permeabilized with 0.1% Triton X-100 for 10 min. HeLa cells were further blocked with 1% BSA at 4° C. overnight, bofore stained with goat anti-human ICAM-1 antibody at a series of dilutions and Alexa Fluor 488-conjugated donkey anti-goat secondary antibodies. Bar represents 10 μm.

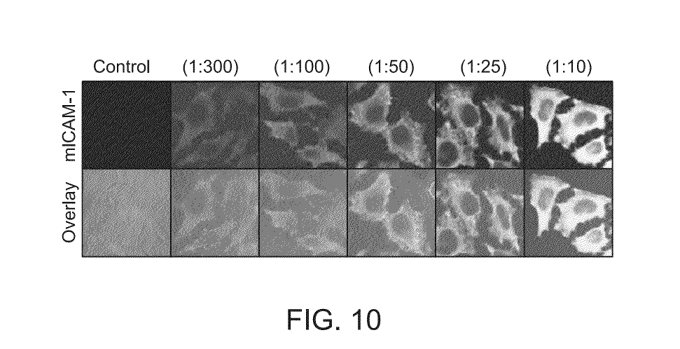

FIG. 11 depicts a graph showing that mICAM-S 1 exhibited significant lacrimal gland distribution after tail intravenous injection to 14 week male NOD mice.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook et al., (1989) Molecular Cloning: A Laboratory Manual, 2nd edition; Ausubel et al., eds. (1987) Current Protocols In Molecular Biology; MacPherson, B. D. Hames and G. R. Taylor eds., (1995) PCR 2: A Practical Approach; Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

The term “pharmaceutically acceptable carrier” (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

The term “therapeutic” refers to an agent or component capable of inducing a biological effect in vivo and/or in vitro. The biological effect may be useful for treating and/or preventing a condition, disorder, or disease in a subject or patient. A therapeutic may include, without limitation, a small molecule, a nucleic acid, or a polypeptide.

As used herein, the term “biological equivalent thereof” is used synonymously with “equivalent” unless otherwise specifically intended. When referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 60%, or 65%, or 70%, or 75%, or 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Alternatively, a biological equivalent is a peptide encoded by a nucleic acid that hybridizes under stringent conditions to a nucleic acid or complement that encodes the peptide or with respect to polynucleotides, those hybridize under stringent conditions to the reference polynucleotide or its complement. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide or polynucleotide region (or a polypeptide or polypeptide region) having a certain percentage (for example, about 60%, 65%, 70%, 75%, 80%, 85%, 90%, 95% or 97%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

An “equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Regulatory polynucleotide sequences” intends any one or more of promoters, operons, enhancers, as known to those skilled in the art to facilitate and enhance expression of polynucleotides.

An “expression vehicle” is a vehicle or a vector, non-limiting examples of which include viral vectors or plasmids, that assist with or facilitate expression of a gene or polynucleotide that has been inserted into the vehicle or vector.

A “delivery vehicle” is a vehicle or a vector that assists with the delivery of an exogenous polynucleotide into a target cell. The delivery vehicle may assist with expression or it may not, such as traditional calcium phosphate transfection compositions.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

As used herein, to “treat” further includes systemic amelioration of the symptoms associated with the pathology and/or a delay in onset of symptoms. Clinical and sub-clinical evidence of “treatment” will vary with the pathology, the subject and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, topical application, intrapentoneal, intravenous and by inhalation. An agent of the present invention can be administered for therapy by any suitable route of administration. It will also be appreciated that the preferred route will vary with the condition and age of the recipient, and the disease being treated.

The agents and compositions of the present invention can be used in the manufacture of medicaments and for the treatment of humans and other animals by administration in accordance with conventional procedures, such as an active ingredient in pharmaceutical compositions. In one aspect, the agents and composition when combined with other therapeutic agents, can be used in the manufacture of combination treatments of humans and other animals by administration in accordance with conventional procedures.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine. In terms of cells, mammalian cells includes, but is not limited to cells of the following origin: a human, a feline, a canine, a simian, a murine, a bovine, an equine, a porcine or an ovine.

As used herein, the term “detectable label” intends a directly or indirectly detectable compound or composition that is conjugated directly or indirectly to the composition to be detected, e.g., N-terminal histadine tags (N-His), magnetically active isotopes, e.g.,¹¹⁵Sn, ¹¹⁷ Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue ™ , and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “elastin-like peptide (ELP) component” intends a polypeptide that forms stable micelle above the transition temperature of the ELP. In one aspect, the ELP component comprises, or alternatively consists essentially of, or yet further consists of the polypeptide S48I48 having the sequence G(VPGSG)n(VPGIG)nY (SEQ ID NO: 4) (wherein n is an integer that denotes the number of repeats, and can be from about 6 to about 192, or alternatively from about 15 to 75, or alternatively from about 40 to 60, or alternatively from about 45 to 55, or alternatively about 48), wherein in one aspect, S48I48 comprises, or alternatively consists essentially of, or yet further consists of the amino acid sequence G(VPGSG)₄₈(VPGIG)₄₈Y (SEQ ID NO: 1), or a biological equivalent thereof. A biological equivalent of polypeptide S48I48 is a peptide that has at least 80% sequence identity to polypeptide S48I48 or a peptide encoded by a polynucleotide that hybridizes under conditions of high stringency to a polynucleotide that encodes polypeptide S48I48 or its complement, wherein conditions of high stringency comprise hybridization reaction at about 60° C. in about 1×SSC. The biological equivalent will retain the characteristic or function of forming a micelle when the biological equivalent is raised above the transition temperature of the biological equivalent or, for example, the transition temperature of S48I48.

ICAM-1 also is known as intercellular adhesion molecule 1 and major group rhinovirus receptor, CD54 antigen. ICAMs are members of the Ig superfamily of calcium-independent transmembrane glycoproteins. ICAM-1 is a ligand for lymphocyte function-associated (LFA) and Mac-1 integrins and the major human rhinovirus receptor. The primary function of ICAM-1 is to provide adhesion between endothelial cells and leukocytes after stress or injury. The human ICAM-1 gene codes for a 505 amino acid transmembrane glycoprotein containing a 29 amino acid cytoplasmic domain, a 23 amino acid transmembrane domain, and a 453 amino acid extracellular domain. Recombinant human ICAM-1 is a 49.5 kDa glycoprotein comprising the extracellular domain (453 amino acid residues) of ICAM-1. Monomeric glycosylated ICAM-1 migrates at an apparent molecular weight of approximately 72.0-80.0 kDa by SDS-PAGE analysis under reducing conditions. The protein is available commercially from PeproTech (Cat. #150-05). A published amino acid sequence is:

(SEQ ID NO: 3)
QTSVSPSKVI LPRGGSVLVT CSTSCDQPKL
LGIETPLPKK ELLLPGNNRK VYELSNVQED SQPMCYSNCP
DGQSTAKTFL TVYWTPERVE LAPLPSWQPV GKNLTLRCQV
EGGAPRANLT VVLLRGEKEL KREPAVGEPA EVTTTVLVRR
DHHGANFSCR TELDLRPQGL ELFENTSAPY QLQTFVLPAT
PPQLVSPRVL EVDTQGTVVC SLDGLFPVSE AQVHLALGDQ
RLNPTVTYGN DSFSAKASVS VTAEDEGTQR LTCAVILGNQ
SQETLQTVTI YSFPAPNVIL TKPEVSEGTE VTVKCEAHPR
AKVTLNGVPA QPLGPRAQLL LKATPEDNGR SFSCSATLEV
AGQLIHKNQT RELRVLYGPR LDERDCPGNW TWPENSQQTP
MCQAWGNPLP ELKCLKDGTF PLPIGESVTV TRDLEGTYLC
RARSTQGEVT RKVTVNVLSP RYE

ICAM-1 has been shown to bind to CD11a, EZR and CD18. CD11a is Integrin, alpha L (antigen CD11A (p180), lymphocyte function-associated antigen 1; alpha polypeptide), also known as ITGAL, is a human gene that functions in the immune system. It is involved in cellular adhesion and costimulatory signaling. It is the target of the drug Efalizumab. Efalizumba (trade name Raptiva, marketed by Genentech, Merck Serono) is used to treat autoimmune diseases such as psoriasis. It is a recombinant humanized monoclonal antibody which acts by inhibiting lymphocyte activation and cell migration out of blood vessels into tissues. ITGAL encodes the integrin alpha L chain. EZR or Ezrin also known as cytovillin or villin-2 is a protein that in humans is encoded by the EZR gene. The cytoplasmic peripheral membrane protein encoded by this gene functions as a protein-tyrosine kinase substrate in microvilli. As a member of the ERM protein family, this protein serves as an intermediate between the plasma membrane and the actin cytoskeleton. It plays a key role in cell surface structure adhesion, migration, and organization. CD18 is also known as integrin beta-2. It is encoded byt the ITGB2 gene. It is reported as the beta subunit of four different structures: LFA-1 (paired with CD11a ); Macrophage-1 antigen (paired with CD11b ); Integrin alphaXbeta2 (paired with CD11c ); and Integrin alphaDbeta2 (paired with CD11i ). The ITGB2 protein product is the integrin beta chain beta 2. Integrins are integral cell-surface proteins composed of an alpha chain and a beta chain. A given chain may combine with multiple partners resulting in different integrins. For example, beta 2 combines with the alpha L chain to form the integrin LFA-1, and combines with the alpha M chain to form the integrin Mac-1. Integrins are known to participate in cell adhesion as well as cell-surface mediated signalling.^{[1]} In humans lack of CD18 causes Leukocyte Adhesion Deficiency, a disease defined by a lack of leukocyte extravasation from blood into tissues.

In one aspect, the polypeptide is mICAM-1 S1, that comprises, or alternatively consists essentially of, or the amino acid sequence FEGFSFLAFEDFVSSI-G(VPGSG)n(VPGIG)nY (SEQ ID NO: 5), wherein “n” denotes the number of repeats and is an integer from about (wherein n is an integer that denotes the number of repeats, and can be from about 6 to about 192, or alternatively from about 15 to 75, or alternatively from about 40 to 60, or alternatively from about 45 to 55, or alternatively about 48) or a biological equivalent thereof, wherein in one aspect, mICAM-1 S1 comprises, or alternatively consists essentially of, or yet further consists of the amino acid sequence FEGFSFLAFEDFVSSI-G(VPGSG)₄₈(VPGIG)₄₈Y (SEQ ID NO: 6) or a biological equivalent thereof. A biological equivalent of polypeptide mICAM-1 S1 is a peptide that has at least 80% sequence identity to polypeptide mICAM-1 S1 or a peptide encoded by a polynucleotide that hybridizes under conditions of high stringency to a polynucleotide that encodes polypeptide mICAM-1 S1 its complement, wherein conditions of high stringency comprise hybridization reaction at about 60° C. in about 1×SSC. The biological equivalent will retain the characteristic or function of binding to the receptor.

In one aspect, the polypeptide hICAM-1 S1 comprises, or alternatively consists essentially of, or the amino acid sequence EWCEYLGGYLRCYA-G(VPGSG)n(VPGIG)nY (SEQ ID NO: 7), wherein “n” denotes the number of repeats and is an integer from about (wherein n is an integer that denotes the number of repeats, and can be from about 6 to about 192, or alternatively from about 15 to 75, or alternatively from about 40 to 60, or alternatively from about 45 to 55, or alternatively about 48), wherein in one aspect, hICAM-1 S1 comprises, or alternatively consists essentially of, or yet further consists of the amino acid sequence EWCEYLGGYLRCYA-G(VPGSg)₄₈(VPGIG)₄₈Y (SEQ ID NO: 8) a biological equivalent thereof. A biological equivalent of polypeptide hICAM-1 S1 is a peptide that has at least 80% sequence identity to polypeptide hICAM-1 S1 or a peptide encoded by a polynucleotide that hybridizes under conditions of high stringency to a polynucleotide that encodes polypeptide hICAM-1 S1 its complement, wherein conditions of high stringency comprise hybridization reaction at about 60° C. in about 1×SSC. The biological equivalent will retain the characteristic or function of binding to the receptor.

### Modes for Carrying Out The Invention

### Abbreviations:

ELP: Elastin-like polypeptide; ICAM-1: Intercellular Adhesion Molecule 1 (CD54: Clusters of Differentiation 54); SI: S48I48 diblock copolymer ELP; mICAM-1 SI: mouse ICAM-1-targeting S48I48 diblock copolymer ELP; hICAM-1 SI: human ICAM-1-targeting S48I48 diblock copolymer ELP; LG: lacrimal gland; LGAC: lacrimal gland acinar cell; APM: apical membrane; BLM: basolateral membrane; WT: wide type; CT-B: Cholera toxin subunit B; CLIC/GEEC: clathrin-independent carrier-GPI-AP-enriched early endosomal compartments.

TABLE 1
Summary of expressed polypeptides
(SEQ ID NOS: 1, 6 and 8, respectively, in order of appearance)

| | | | | Expected | Measured | Hydrodynamic |
|---|---|---|---|---|---|---|
| Peptide | | CMT | bulk | molecular | molecular | radius (nm) |
| label | Amino acid sequence | (° C.)^{a} | (° C.)^{b} | weight (kDa) | weight (kDa)^{c} | at 37° C.^{d} |
| S48I48 | G(VPGSG)₄₈(VPGIG)₄₈Y | 26.5 | 76.0 | 39.64 | 39.54 | 23.47 |
| mICAM-1 SI | FEGFSFLAFEDFVSSI- | 25.7 | 46.9 | 41.48 | 41.42 | 23.8 |
| | G(VPGSG)₄₈(VPGIG)₄₈Y | | | | | |
| hICAM-1 SI | EWCEYLGGYLRCYA- | 19.1 | 23.8 | 41.37 | 41.22 | — |
| | G(VPGSG)₄₈(VPGIG)₄₈Y | | | | | |

^{a}Critical micelle temperature and ^{b}Bulk transition temperature were measured using optical density at 350 nm with a UV-Vis spectrophotometer.
^{c}Molecular weight was determined using MALDI-TOF mass spectrometry.
^{d}Hydrodynamic radius was determined using dynamic light scattering.
The subscript denotes the number of repeats.

This disclosure provides a protein-based targeted drug delivery product and method that specifically suppresses the autoinflammatory diseases of the human lacrimal gland, including Sjögren's Syndrome (SjS). This treatment uses protein polymeric nanoparticles to specifically target a molecular marker of the inflamed lacrimal gland epithelium; furthermore, the nanoparticles, in one aspect, further comprise therapeutic agents. Non-limiting examples include without limitation a peptide-based inhibitor of the cathepsin S protease, which is implicated in the pathological inflammation of the ocular surface system associated with SjS. The nanoparticles comprises a genetically engineered elastin-like polypeptide (ELP) protein polymers that assemble into nanoparticles (S48I48) to contain peptides that binding to ICAM-1 moieties (anti-murine mICAM-1 SI, anti-human hICAM-1 SI). In another aspect, the nanoparticles further comprises peptidic protease inhibitors (e.g., cathepsin S inhibitory peptides), small molecules, or immunosuppressants for treating or ameliorating the symptoms of SjS. Thus, this disclosure also provides methods to treat or ameliorate the symptoms of SjS and associated disorders (e.g., autoimmune disorders) in a disease progression-oriented manner by targeting ICAM-1 receptors that are overexpressed on the surface of diseased lacrimal gland acinar cells. Since ICAM-1 receptor itself is internalized by endocytosis following ligand binding, this receptor is also an ideal target internalization of the targeted nanoparticles to the interior of the diseased acinar cells. The compositions also are useful to treat or ameliorate the symptoms of other autoimmune diseases, non-limiting examples of which include rheumatoid arthritis and systemic lupus erythematosus, or diseases eventually resulting in enhanced expression of ICAM-1 receptors. To date, there is no effective cure for SjS, and this invention is the first to offer a practical approach for treating this disease by selectively delivering drugs into lysosomes and secretory vesicles of diseased cells. Moreover, by employing nanotechnology for the drug delivery system, this invention offers potential opportunities to target the therapeutic effect with high specificity, long residence time, and low systemic side effects.

It Applicants' belief that this is first disclosure of an approach has been proposed to deliver cathepsin S inhibitors selectively into lysosomes and secretory vesicles of diseased LGACs for treating SjS.

It also is the Applicants' belief that this the first disclosure of protein polymers for use as a carrier for short peptide-inhibitors of known proteases. Short peptides have limited pharmacokinetic properties, which prevents them from being successful drugs. Attachment to targeted or untargeted protein polymers offers a unique solution to overcome this limitation, which could have broad implications in the delivery of other peptide-inhibitors of proteases.

It is the Applicants' belief that mICAM-1 SI protein polymers offer the selectively targeted treatment with high specificity, long residence time, and low dose-dependent systemic side effects.

It is also the Applicants' belief that this is the first disclosure of protein polymer nanoparticles that are targeted to inflammation via the ICAM-1 receptor. This receptor plays a role in diseases beyond that of the ocular surface system, including cardiovascular disease.

Elastin-like polypeptides (ELPs)

Elastin-like-polypeptides (ELPs) are a genetically engineered polypeptide with unique phase behavior (see for e.g., S. R. MacEwan, et al., Biopolymers 94(1) (2010) 60-77) which promotes recombinant expression, protein purification and self-assembly of nanostructures (see for e.g., A. Chilkoti, et al., Advanced Drug Delivery Reviews 54 (2002) 1093-1111). ELPs are artificial polypeptides composed of repeated pentapeptide sequences, (Val-Pro-Gly-Xaa-Gly)n (SEQ ID NO: 9) derived from human tropoelastin, where Xaa (also denoted “X” herein) is the “guest residue” which is any amino acid. In one embodiment, Xaa is any amino acid except proline. This peptide motif displays rapid and reversible de-mixing from aqueous solutions above a transition temperature, Tₜ. Below Tₜ, ELPs adopt a highly water soluble random coil conformation; however, above Tₜ, they separate from solution, coalescing into a second aqueous phase. The Tₜ of ELPs can be tuned by choosing the guest residue and ELP chain length as well as fusion peptides at the design level (see for e.g., MacEwan S R, et al., Biopolymers 94(1): 60-77). The ELP phase is both biocompatible and highly specific for ELPs or ELP fusion proteins, even in complex biological mixtures. Genetically engineered ELPs are monodisperse, biodegradable, non-toxic. Throughout this description, ELPs are identified by the single letter amino acid code of the guest residue followed by the number of repeat units, n. For example, in one aspect, the ELP is the peptide S48I48 which represents a diblock copolymer ELP with 48 serine (S) pentamers at the amino terminus and 48 isoleucine (I) pentamers at the carboxy terminus.

Described herein are ELP fusion proteins, which can be self-assembled into nanoparticles. The diameter of the nanoparticle can be from about 1 to about 1000 nm or from about 1 to about 500 nm, or from about 1 to about 100 nm, or from about 1 to about 50 nm, or from about 20 to about 50 nm, or from about 30 to about 50 nm, or from about 35 to about 45 nm. In one embodiment, the diameter is about 40 nm. These nanoparticles can be high efficiently internalized into LGAC. The fusion proteins are composed of elastin-like-polypeptides and high affinity polypeptides. These fusion proteins can be expressed from a variety of expression systems known to those skilled in the art and easily purified by the phase transition behavior of ELPs. These ELP fusion proteins are able to conjugate small molecules, such as, for example, chemotherapeutic agents, anti-inflammation agents, antibiotics and polypeptides and other water soluble drugs. In addition, the ELP nanoparticles are useful for carrying DNA, RNA, protein and peptide-based therapeutics.

ELPs have potential advantages over chemically synthesized polymers as drug delivery agents. First, because they are biosynthesized from a genetically encoded template, ELPs can be made with precise molecular weight. Chemical synthesis of long linear polymers does not typically produce an exact length, but instead a range of lengths. Consequently, fractions containing both small and large polymers yield mixed pharmacokinetics and biodistribution. Second, ELP biosynthesis produces very complex amino acid sequences with nearly perfect reproducibility. This enables very precise selection of the location of drug attachment. Thus drug can be selectively placed on the corona, buried in the core, or dispersed equally throughout the polymer. Third, ELP can self-assemble into multivalent nanoparticles that can have excellent site-specific accumulation and drug carrying properties. Fourth, because ELP are designed from native amino acid sequences found extensively in the human body they are biodegradable, biocompatible, and tolerated by the immune system. Fifth, ELPs undergo an inverse phase transition temperature, Tₜ, above which they phase separate into large aggregates. By localized heating, additional ELP can be drawn into the target site, which may be beneficial for increasing drug concentrations.

A therapeutic such as a drug, for example, may be attached to the ELP through cysteine, lysine, glutamic acid or aspartic acid residues present in the polymer. In some embodiments, the cysteine, lysine, glutamic acid or aspartic acid residues are generally present throughout the length of the polymer. In some embodiments, the cysteine, lysine, glutamic acid or aspartic acid residues are clustered at the end of the polymer. In some embodiments of the presently described subject matter, therapeutics are attached to the cysteine residues of the ELP using thiol reactive linkers, a cleavable disulfide linker, a hydrophilic flexible linker comprised of amino acids (GGGGS)ₙ(SEQ ID NO: 10) (wherein n is an integer from 1 to 5, and preferably 3) or a rigid linker comprised of amino acids (EAAAK₁₀(SEQ ID NO: 11) (wherein n is an integer from 1 to 5, and preferably 3). In some embodiments of the presently described subject matter, therapeutics are attached to the lysine residues of the high molecular weight polymer sequence using NHS (N-hydroxysuccinimide) chemistry to modify the primary amine group present on these residues. In some embodiments of the presently described subject matter, therapeutics are attached to the glutamic acid or aspartic acid residues of the ELP using EDC (1-Ethyl-3-[3-dimethylaminopropyl]carbodiimide Hydrochloride) chemistry to modify the carboxylic acid group present on the ELP residues.

The therapeutic associated with the ELP may be hydrophobic or hydrophilic. Which the drug is hydrophobic, attachment to the terminus of the ELP may facilitate formation of the multivalent nanoparticle. The number of drug particles attached to the ELP can be from about 1 to about 30, or from about 1 to about 10, or about 1, 2, 3, 4, 5, 6, 7, 8, 9, or 10. In some embodiments, the attachment points for a therapeutic are equally distributed along the backbone of the ELP, and the resulting drug-ELP is prevented from forming nanoparticle structures under physiological salt and temperature conditions.

In addition to therapeutics, the ELPs may also be associated with a detectable label that allows for the visual detection of in vivo uptake of the ELPs. Suitable labels include, for example, fluorescein, rhodamine, tetramethylrhodamine, eosin, erythrosin, coumarin, methyl-coumarins, pyrene, Malacite green, Alexa-Fluor®, stilbene, Lucifer Yellow, Cascade Blue™, and Texas Red. Other suitable optical dyes are described in Haugland, Richard P. (1996) Molecular Probes Handbook.

In certain embodiments, the ELP components include polymeric or oligomeric repeats of the pentapeptide (VPGXG)n (SEQ ID NO: 12), wherein n is an integer representing the number of repeats between 5 and 400, alternatively between 5 and 300, or alternatively between 25 and 250, or alternatively between 25 and 150, where the guest residue X is any amino acid, that in one aspect, excludes proline. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine.

The guest residue X may be a non-classical (non-genetically encoded) amino acid. Examples of non-classical amino acids include: D-isomers of the common amino acids, 2, 4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine,β-alanine, fluoro-amino acids, designer amino acids such as β-methyl amino acids, C α-methyl amino acids, N α-methyl amino acids, and amino acid analogs in general.

Selection of X is independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

The ELP component in some embodiments is selected or designed to provide a Tₜ ranging from about 10 to about 80° C., such as from about 35 to about 60° C., or from about 38 to about 45° C. In some embodiments, the Tₜ is greater than about 40° C. or greater than about 42° C., or greater than about 45° C., or greater than about 50° C. The transition temperature, in some embodiments, is above the body temperature of the subject or patient (e.g., >37° C.) thereby remaining soluble in vivo, or in other embodiments, the Tₜ is below the body temperature (e.g., <37° C.) to provide alternative advantages, such as in vivo formation of a drug depot for sustained release of the therapeutic agent.

The Tₜ of the ELP component can be modified by varying ELP chain length, as the Tₜ generally increases with decreasing MW. For polypeptides having a molecular weight >100,000, the hydrophobicity scale developed by Urry et al. (PCT/US96/05186, which is hereby incorporated by reference in its entirety) is preferred for predicting the approximate Tₜ of a specific ELP sequence. However, in some embodiments, ELP component length can be kept relatively small, while maintaining a target Tₜ, by incorporating a larger fraction of hydrophobic guest residues (e.g., amino acid residues having hydrophobic side chains) in the ELP sequence. For polypeptides having a molecular weight <100,000, the Tₜ may be predicted or determined by the following quadratic function: Tₜ=M₀+M₁X+M₂X² where X is the MW of the fusion protein, and M₀=116.21; M₁=−1.7499; M₂=0.010349.

While the Tₜ of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, and half-life of the molecule.

### Ligands

The ligands are selected for targeting the mammalian ICAM-1 receptor. In certain aspects, either mouse ICAM-1 targeting peptides, FEGFSFLAFEDFVSSI (SEQ ID NO: 13), or human ICAM-1 targeting peptides, EWCEYLGGYLRCYA (SEQ ID NO: 14), are conjugated to the N-terminus of SI DNA fragments. See experimental section below. However, biological equivalents of the peptides are also within the scope of this disclosure, e.g., peptides having at least 80% sequence identity and the ability to selectively bind ICAM-1 using methods known in the art. Biological equivalents are include peptides encoded by polynucleotides that hybridize under conditions of high stringency to a reference polypeptide that encodes FEGFSFLAFEDFVSSI (SEQ ID NO: 13) or EWCEYLGGYLRCYA (SEQ ID NO: 14) or their complements and retain the ability to selectively bind a ICAM-1 receptor. Conditions of high stringency and methods to determine sequence identity are disclosed herein and known in the art.

### Expression of Recombinant Proteins

ELPs and other recombinant proteins described herein can be prepared by expressing polynucleotides encoding the polypeptide sequences of this invention in an appropriate host cell, i.e., a prokaryotic or eukaryotic host cell This can be accomplished by methods of recombinant DNA technology known to those skilled in the art. It is known to those skilled in the art that modifications can be made to any peptide to provide it with altered properties. Polypeptides of the invention can be modified to include unnatural amino acids. Thus, the peptides may comprise D-amino acids, a combination of D- and L-amino acids, and various “designer” amino acids (e.g.,β-methyl amino acids, C-α-methyl amino acids, and N-a-methyl amino acids, etc.) to convey special properties to peptides. Additionally, by assigning specific amino acids at specific coupling steps, peptides with α-helices, βturns, βsheets, α-turns, and cyclic peptides can be generated. Generally, it is believed that beta-turn spiral secondary structure or random secondary structure is preferred. Accordingly, polynucleotides encoding the ELPs of this disclosure are provided herein, including the polynucleotides contained within a vector and/or host cell system, as well as methods for recombinantly or chemically synthesizing the ELPs and then further isolating the ELP.

The ELPs can be expressed and purified from a suitable host cell system. Suitable host cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In one embodiment, the host cell is E. coli. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Protein Purification

The phase transition behavior of the ELPs allows for easy purification. The ELPs may also be purified from host cells using methods known to those skilled in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC. In the case of ELP compositions protein purification may also be aided by the thermal transition properties of the ELP domain as described in U.S. Pat. No. 6,852,834.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “[n]-fold purification number” wherein n is an integer. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

### Pharmaceutical Compositions

Pharmaceutical compositions are further provided. The compositions comprise a carrier and ELPs as described herein. The carriers can be one or more of a solid support or a pharmaceutically acceptable carrier. In one aspect, the compositions are formulated with one or more pharmaceutically acceptable excipients, diluents, carriers and/or adjuvants. In addition, embodiments of the compositions include ELPs, formulated with one or more pharmaceutically acceptable auxiliary substances.

The invention provides pharmaceutical formulations in which the one or more of an isolated agent of the invention, an isolated polypeptide of the invention, an isolated polynucleotide of the invention, a vector of the invention, an isolated host cell of the invention, or an antibody of the invention can be formulated into preparations for injection in accordance with the invention by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g., a Type IV Pilin protein (see Jurcisek and Bakaletz (2007) J. of Bacteriology 189(10):3868-3875) and antibacterial agents.

Aerosol formulations provided by the invention can be administered via inhalation. For example, embodiments of the pharmaceutical formulations of the invention comprise a compound of the invention formulated into pressurized acceptable propellants such as dichlorodifluoromethane, propane, nitrogen and the like.

Embodiments of the pharmaceutical formulations of the invention include those in which the ELP is formulated in an injectable composition. Injectable pharmaceutical formulations of the invention are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the invention.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the compound adequate to achieve the desired state in the subject being treated.

Routes of administration applicable to the methods and compositions described herein include intranasal, intramuscular, subcutaneous, intradermal, topical application, intravenous, nasal, oral, inhalation, intralacrimal, retrolacrimal profusal along the duct, intralacrimal, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In one embodiment, the composition comprising the ELP is administered intralacrimally through injection. In further embodiments, the composition is administered systemically, topically on top of the eye, by retrolacrimal profusion, or intranasally.

### Treatment of Disease

Methods and compositions disclosed herein are useful ameliorating the symptoms of disease for treating disease. Non-limiting examples of such include cancer, an autoimmune disease, age-related macular degeneration, Sjögren's syndrome, autoimmune exocrinopathy, diabetic retinopathy, graft versus host disease, exocrinopathy, retinal venous occlusions, retinal arterial occlusion, macular edema, postoperative inflammation, uveitis retinitis, proliferative vitreoretinopathy, glaucoma, keratoconjunctivitis sicca (dry eye), scleritis or glaucoma.

In one aspect, the agent treats or ameliorates symptoms of diseases or disorders of the eye. The lacrimal gland acinar cell targeted ELPs provide a site-specific target therapeutic. Accordingly, these ELP nanoparticles may be useful to encapsulate or attach drugs for treating disorders localized to the eye. By way of example, these disorders can include, age-related macular degeneration, Sjögren's syndrome, autoimmune exocrinopathy, diabetic retinopathy, graft versus host disease (exocrinopathy associated with) retinal venous occlusions, retinal arterial occlusion, macular edema, postoperative inflammation, uveitis retinitis, proliferative vitreoretinopathy and glaucoma. In one embodiment, the disease is Sjögren's syndrome. In another embodiment, the disease is keratoconjunctivitis sicca (dry eye). In another embodiment the disease is scleritis. In another embodiment the disease is glaucoma.

### Combination Treatments

Administration of the therapeutic agent or substance of the present invention to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy.

As is apparent to those skilled in the art, the combination therapy can take the form of a combined therapy for concurrent or sequential administration.

### Kits

The ELPs as described herein, can be provided in kits. The kits can further contain additional therapeutics and optionally, instructions for making or using the ELPs. In a further aspect, the kit contains reagents and instructions to perform a screen as detailed herein.

### Screening Assays

This invention also provides screening assays to identify potential therapeutic agents of known and new compounds and combinations. For example, one of skill in the art can also determine if the ELP provides a therapeutic benefit in vitro by contacting the ELP or combination comprising the ELP with a sample cell or tissue to be treated. The cell or tissue can be from any species, e.g., simian, canine, bovine, ovine, rat, mouse or human.

The contacting can also be performed in vivo in an appropriate animal model or human patient. When performed in vitro, the ELPs can be directly added to the cell culture medium. When practiced in vitro, the method can be used to screen for novel combination therapies, formulations or treatment regimens, prior to administration to an animal or a human patient.

In another aspect, the assay requires contacting a first sample comprising suitable cells or tissue (“control sample”) with an effective amount of an ELP as disclosed herein and contacting a second sample of the suitable cells or tissue (“test sample”) with the ELP, agent or combination to be assayed. In one aspect in the case of cancer, the inhibition of growth of the first and second cell samples are determined. If the inhibition of growth of the second sample is substantially the same or greater than the first sample, then the agent is a potential drug for therapy. In one aspect, substantially the same or greater inhibition of growth of the cells is a difference of less than about 1%, or alternatively less than about 5% or alternatively less than about 10% , or alternatively greater than about 10% , or alternatively greater than about 20%, or alternatively greater than about 50%, or alternatively greater than about 90%. The contacting can be in vitro or in vivo. Means for determining the inhibition of growth of the cells are well known in the art.

In a further aspect, the test agent is contacted with a third sample of cells or tissue comprising normal counterpart cells or tissue to the control and test samples and selecting agents that treat the second sample of cells or tissue but does not adversely affect the third sample. For the purpose of the assays described herein, a suitable cell or tissue is described herein such as cancer or other diseases as described herein. Examples of such include, but are not limited to cancer cell or tissue obtained by biopsy, blood, breast cells, colon cells.

Efficacy of the test composition is determined using methods known in the art which include, but are not limited to cell viability assays or apoptosis evaluation.

In yet a further aspect, the assay requires at least two cell types, the first being a suitable control cell.

The assays also are useful to predict whether a subject will be suitably treated by this invention by delivering an ELP to a sample containing the cell to be treated and assaying for treatment which will vary with the pathology or for screening for new drugs and combinations. In one aspect, the cell or tissue is obtained from the subject or patient by biopsy. This disclosure also provides kits for determining whether a pathological cell or a patient will be suitably treated by this therapy by providing at least one composition of this invention and instructions for use.

The test cells can be grown in small multi-well plates and is used to detect the biological activity of test compounds. For the purposes of this invention, the successful ELP or other agent will block the growth or kill the cancer cell but leave the control cell type unharmed.

### Experimental

Preparation of ICAM-1 ELPs and cathepsin S inhibitory peptides

In the ICAM project, Applicants produced mouse ICAM-1 targeting SI, mICAM-1 SI, and human ICAM-1 targeting SI, hICAM-1 SI, via the recursive directional ligation (RDL) and inverse transition cycling (ITC). In brief, the recombinant plasmid encoding the sequence G(VPGSG)₄₈(VPGIG)₄₈Y (SEQ ID NO: 1), (abbreviated as SI, and wherein the subscript “48” represents the number of repeats), was constructed using RDL, and followed by conjugating either mouse ICAM-1 targeting peptides, FEGFSFLAFEDFVSSI (SEQ ID NO: 13), or human ICAM-1 targeting peptides, EWCEYLGGYLRCYA (SEQ ID NO: 14), to the N-terminus of SI DNA fragments. The resulting plasmids were transformed into BLR competent cells to express mICAM-1 SI and hICAM-1 SI, respectively. The purification of ELPs was followed as described herein. Sequences of mouse and human ICAM-1 targeting peptide were identified by other groups (Belizaire, A. K. et al. Biochemical and Biophysical Research Communications. 309, 625-630(2003) and Welply, J. K. et al. A peptide isolated by phage display binds to ICAM-1 and inhibits binding to LFA-1. Protein: structure, function, and genetics. 26, 262-270 (1996)) and obtained using the phage display technology. Cathepsin S inhibitory peptides (CATSIP), NHLGDMTSEEVMSLTSS (SEQ ID NO: 2), can be fused to the N-terminus of SI DNA fragments used to express CATSIP-SI. CATSIP is a fragment derived from rat cathepsin pro-peptides and exhibited enzymatic activities against human and mouse cathepsin S. As is apparent to the skilled artisan, other therapeutic peptides can be fused to the ELPs independently or with the cathepsin S.

Applicants synthesized mICAM-1 SI and hICAM-1 SI, obtained their Tt profiles, and confirmed that mICAM-1 SI self-assembles into multivalent and monodisperse micelles with a hydrodynamic diameter of 45 nm. The size of mICAM-1 SI was further verified by TEM and cryo-TEM. mICAM-1 SI exhibited enhanced internalization in both mICAM-1 transfected and non-transfected HeLa cells expressing ICAM receptors compared to plain ELP, SI. The mRNA levels of mICAM-1 receptor were significantly elevated (˜sevenfold) in the lacrimal glands of NOD mice, specifically at the basolateral membrane of acinar cells, compared with expression in lacrimal glands of disease-free BALB/c mice.

### Intravenous Administration of ELPs

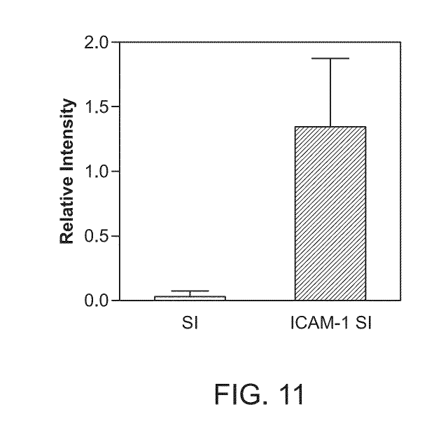

Male NOD mice aged 12-14 weeks were used for animal studies. For tail vein injection of Rhodamine-labeled SI and mICAM-1 SI, the mice were administered 100 μl of 200 μM rhodamine-labeled SI or mICAM-1 SI twice, with 1 hr time interval, via tail intravenous injection. After lhr of the second injection, the mice were anesthetized by intraperitoneal (IP) injection with ketamine and xylazine. The LGs were removed, placed in OCT, snap frozen in liquid nitrogen, cut into 10-82 m -thick sections mounted to objective slides, and stored in−80° C. prior to imaging using confocal microscope. For intralacrimal gland injection of Rhodamine-labeled SI and mICAM-1 SI, the mice were anesthetized via IP injection of ketamine and xylazine. A small incision was made on an axis between the ear and eye to expose LGs. The LGs were injected with 5 μl of 50 μM Rhodamine-labeled SI and 50 μg CF-dextran 10K or 5 μl of 50 μM Rhodamine-labeled mICAM-1 SI and 50 μg CF-dextran 10 K directly. After 1-hr incubation, The LGs were removed, placed in OCT, snap frozen in liquid nitrogen, cut into 10-μm-thick sections mounted to objective slides, and stored in −80° C. prior to imaging using confocal microscope (data not shown). The sections were examined with a confocal laser scanning microscope (LSM) at excitation wavelengths of 488 and 534 nm. Quantification of internalized SI or mICAM-1 SI inside LG clusters was measured using ImageJ (See FIG. 11). Data were analyzed by student's t test and presented as Mean±SEM (SI: 0.03289±0.01834; mICAM-1 SI: 1.348±0.2346).p=0.0005. N=5.

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention such as for example, embodiments described in Appendix A attached hereto. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. An agent comprising an elastin-like peptide (ELP) component that forms a stable micelle above the transition temperature of the ELP and a ligand that binds a mammalian ICAM-1 receptor.

2. The agent of claim 1, further comprising a therapeutic agent.

3. The agent of claim 2, wherein the therapeutic agent comprises a cathepsin S inhibitory peptide (CATSIP) of the reference sequence NHLGDMTSEEVMSLTSS or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC.

4. The agent of any one of claims 1-3, wherein the therapeutic agent is trapped within a stable micelle formed by the ELP when the ELP is above the transition temperature of the ELP.

5. The agent of any one of claims 1-3, further comprising a linker between the ligand and the ELP.

6. The agent of claim 1, wherein the ELP comprises:
a. the reference polypeptide S48I48 (G(VPGSG)n(VPGIG)nY (SEQ ID NO: 4) (wherein n is an integer that denotes the number of repeats, and can be from about 6 to about 192, or alternatively from about 15 to 75, or alternatively from about 40 to 60, or alternatively from about 45 to 55, or alternatively about 48)) or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1 ×SSC; and
b. the ligand that binds a mammalian ICAM-1 receptor comprises a murine ICAM-1 receptor or a human ICAM-1 receptor.

7. The agent of claim 6, wherein the ligand that binds mammalian ICAM-1 receptor comprises a reference polypeptide sequence of the group:
a. FEGFSFLAFEDFVSSI (SEQ ID NO: 13) or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC, or
b. EWCEYLGGYLRCYA (SEQ ID NO: 14) or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC.

8. The agent of claim 7, wherein the agent comprises a polypeptide of the group:
a. a polypeptide having the reference sequence FEGFSFLAFEDFVSSI-G(VPGSG)n(VPGIG)nY (SEQ ID NO: 5), wherein “n” denotes the number of repeats and is an integer from about (wherein n is an integer that denotes the number of repeats, and can be from about 6 to about 192, or alternatively from about 15 to 75, or alternatively from about 40 to 60, or alternatively from about 45 to 55, or alternatively about 48, or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC; or
b. a polypeptide having the reference sequence EWCEYLGGYLRCYA-G(VPGSG)n(VPGIG)nY (SEQ ID NO: 7), wherein “n” denotes the number of repeats and is an integer from about (wherein n is an integer that denotes the number of repeats, and can be from about 6 to about 192, or alternatively from about 15 to 75, or alternatively from about 40 to 60, or alternatively from about 45 to 55, or alternatively about 48, or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC.

9. The agent of claim 1, wherein the agent comprises the polypeptide sequence of the group:
a. a polypeptide having the reference sequence FEGFSFLAFEDFVSSI-G(VPGSG)₄₈(VPGIG)₄₈Y (SEQ ID NO: 6), wherein “48” denotes the number of repeats, or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC; or
b. a polypeptide having the reference sequence EWCEYLGGYLRCYA-G(VPGSG)₄₈(VPGIG)₄₈Y (SEQ ID NO: 8), wherein “48” denotes the number of repeats, or a biological equivalent thereof, wherein a biological equivalent of the reference peptide is a peptide that has at least 80% sequence identity to the reference sequence or a peptide encoded by a polynucleotide that hybridizes under conditions of high stringency to a polynucleotide that encodes the reference peptide or its complement, wherein conditions of high stringency comprise hybridization reaction at about 60° C. in about 1×SSC.

10. The agent of claim 3, wherein the therapeutic agent is directed to the treatment or amelioration of one or more of cancer, an autoimmune disease, age-related macular degeneration, Sjögren's syndrome, autoimmune exocrinopathy, diabetic retinopathy, graft versus host disease, exocrinopathy, retinal venous occlusions, retinal arterial occlusion, macular edema, postoperative inflammation, uveitis retinitis, proliferative vitreoretinopathy, glaucoma, keratoconjunctivitis sicca (dry eye), scleritis or glaucoma.

11. The agent of claim 10, wherein the therapeutic agent is one or more of pilocarpin, cevimeline, ophthalmic cyclosporine, nonsteriodal anti-inflammatory drugs (NSAIDs) and androgen analogues.

12. The agent of claim 1, further comprising a detectable label.

13. A composition comprising the agent of claim 1 and a carrier.

14. The composition of claim 13, wherein the carrier is a pharmaceutically acceptable carrier.

15. A method for delivering a drug in vitro comprising contacting a tissue expressing an ICAM-1 receptor with the agent of claim 1.

16. A method for delivering a drug in vivo comprising administering an effective amount of the agent of claim 1 to a subject.

17. A method for ameliorating the symptoms of a disease or condition or for treating a disease or condition, the method comprising administering an effective amount of the agent of any one of claim 2, 3, 6, 9 or 10 to a subject suffering from the disease or condition or susceptible to the disease or condition.

18. A method for ameliorating the symptoms of a disease or condition selected from one or more of cancer, an autoimmune disease, age-related macular degeneration, Sjögren's syndrome, autoimmune exocrinopathy, diabetic retinopathy, graft versus host disease, exocrinopathy, retinal venous occlusions, retinal arterial occlusion, macular edema, postoperative inflammation, uveitis retinitis, proliferative vitreoretinopathy, glaucoma, keratoconjunctivitis sicca (dry eye), scleritis or glaucoma, comprising administering an effective amount of an agent of claim 2 to a subject suffering from the disease or condition.

19. A kit for treating for ameliorating the symptoms of a disease or condition or for treating a disease or condition, the kit comprising a an effective amount of the agent of claim 1 and instructions for use.

20. An isolated polynucleotide encoding an agent of claim 1 and optionally being operatively linked to a regulatory or expression sequence.

21. An isolated vector and/or host cell comprising the isolated polynucleotide of claim 20.

22. A composition comprising the isolated polynucleotide of claim 20 and a carrier.

23. A method for recombinantly producing a therapeutic agent comprising expressing the polynucleotide of claim 20 under conditions that favor expression of the polynucleotide.

24. The method of claim 23, further comprising isolating the therapeutic agent.

25. The method of claim 23, further comprising preparing a composition comprising the therapeutic agent and the ELP-fusion and subsequently raising the temperature of the above the transition temperature of the ELP.
